(19) United States
(12) Patent Application Publication
(10) Pub. No.: US 2023/0065759 A1
(43) Pub. Date: Mar. 2, 2023

(54) AUTOMATED MANAGEMENT OF CARE RECIPIENT TREATMENT REGIMENS

(71) Applicant: INTERNATIONAL BUSINESS MACHINES CORPORATION, Armonk, NY (US)

(72) Inventors: David Bacarella, Naperville, IL (US); Clinton Anthony Black, Oak Park, IL (US); Natalie Brooks Powell, Bolingbrook, IL (US); ARIS GKOULALAS-DIVANIS, Waltham, MA (US); Susan Hallen, Elk Grove Vlllage, IL (US); Pranay Kumar Jha, New Delhi (IN); Elisabetta Rinaldi, Roma (IT); Akila Srinivasan, Carpentersville, IL (US)

(21) Appl. No.: 17/446,721

(22) Filed: Sep. 2, 2021

(51) Int. Cl. G16H 50/30; G16H 50/20; G16H 10/60; G16H 20/10; G16H 50/70; G16H 40/67; A61B 5/16

(52) U.S. Cl. CPC G16H 50/30; G16H 50/20; G16H 10/60; G16H 20/10; G16H 50/70; G16H 40/67; A61B 5/165

(57) ABSTRACT

A computer-implemented method, a computer system and a computer program product manage a medical treatment of a care recipient. The method includes obtaining a treatment record for the care recipient from a server. The method also includes capturing real-time biometric data of the care recipient using a plurality of sensors. The method further includes determining a severity level of the adverse event by comparing the real-time biometric data with the treatment record in response to detecting an adverse event in the real-time biometric data. In addition, the method includes generating a recommendation for an alternative treatment in response to the severity level being above a threshold. Lastly, the method includes transmitting a notification comprising the recommendation to a treatment provider.

### BACKGROUND

Embodiments relate generally to providing enhanced health care services, and more specifically to automating the management of a care recipient treatment regimen.

Treatments, including medication regimens, may be administered to care recipients for a variety of reasons and conditions. Detailed medical records may be kept about care recipients and their treatment plans, including medication regimens, for use in tracking an effectiveness of treatment, as well as documenting side effects of a particular medication regimen, for an individual care recipient. The information in these medical records may be useful in managing treatment and determining if a treatment plan or medication regimen may be altered for an individual care recipient, as well as anonymous documentation of adverse effects of specific medication regimens for the public at large.

### SUMMARY

An embodiment is directed to a computer-implemented method for managing a medical treatment of a care recipient. The method may include obtaining a treatment record for the care recipient from a server. The method also may include capturing real-time biometric data of the care recipient using a plurality of sensors. The method may further include determining a severity level of the adverse event by comparing the real-time biometric data with the treatment record in response to detecting an adverse event in the real-time biometric data. In addition, the method may include generating a recommendation for an alternative treatment in response to the severity level being above a threshold. Lastly, the method may include transmitting a notification comprising the recommendation to a treatment provider.

In another embodiment, the method may include receiving a plurality of care recipient agents. Each care recipient agent may include a treatment record of a respective care recipient. The method may also include selecting a care recipient agent from the plurality of care recipient agents by comparing the real-time biometric data and the treatment record of the care recipient to the treatment record of each care recipient agent. Lastly, the method may include placing the selected care recipient agent in a group of care recipient agents.

In a further embodiment, capturing the real-time biometric data may include using a machine learning model that predicts a user's emotions from the captured biometric data to determine an emotional state of the care recipient.

In still another embodiment, determining the severity level of the adverse event may include comparing the captured real-time biometric data and the treatment record of the care recipient to the treatment record of other care recipient agents within the group of care recipient agents.

In yet another embodiment, transmitting the notification may include updating the treatment record of the care recipient with the alternative treatment.

In another embodiment, the alternative treatment may include a change to a medication currently used in the medical treatment.

In a further embodiment, the alternative treatment may include a change to a current administration of the medical treatment.

In addition to a computer-implemented method, additional embodiments are directed to a system and a computer program product for managing a medical treatment of a care recipient.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 depicts a block diagram of an example computer system in which various embodiments may be implemented.

FIG. 2 depicts a block diagram of a computing system that may be used to create a group of care recipient agents for the purpose of sharing information about treatment between care recipients, according to an exemplary embodiment.

FIG. 3 depicts a flow chart diagram of a process to manage care recipient treatment options, including medication regimens, according to an embodiment.

FIG. 4 depicts a cloud computing environment according to an embodiment.

FIG. 5 depicts abstraction model layers according to an embodiment.

### DETAILED DESCRIPTION

Medical professionals may prescribe treatment options, including medication regimens, to their patients, or care recipients, for a variety of reasons and conditions. In many cases, treatment options may be well-known, including side effects of various medications. For instance, medications related to cholesterol or heart disease may also carry side effects related to joint or muscle pain. Knowledge of these side effects may be used by the medical professional to adjust treatment as needed to minimize discomfort of the care recipient. However, in some cases, a treatment option may be less understood, such as where a medication may be newly released to the public or if the care recipient has specific vulnerabilities that are unique and have not been reported in the general medical literature. As a result, care recipients may needlessly suffer from the side effects of a medication regimen. In extreme cases, a medication regimen may compromise a care recipient's health in unforeseen ways and potentially threaten their lives. This may be especially concerning in care recipients that suffer from dementia or other conditions where a care recipient is either unaware of or cannot express if they are experiencing problems related to their medical treatment.

What is needed is a method for automating the management of treatment options, such as medication regimens, in a way that information may be shared across the community and may be used by a network of care recipient agents that may be aware of known problems, or adverse events, and also may be able to react quickly to adverse events that are not anticipated and generate recommendations for alternative treatments. Such care recipient agents may leverage physiological information, i.e., biometric data, collected from sensors monitoring a care recipient and, if permission has been granted, alert appropriate medical professionals to alternative treatments that may better suit the care recipient.

To accomplish this, embodiments may send collected information about adverse events experienced by a care recipient to a group, or ad-hoc network, of agents that may be treating similar care recipients, with the intent of discovering the same or similar adverse events in these care recipients. This can be very useful especially in situations where a particular adverse event may be previously unknown or of different intensity (e.g., headache, nausea, etc.)

Once an appropriate network of agents is established, the severity of an adverse event may be quantitatively assessed by comparing the captured biometric data with information gathered from the network of care recipient agents, paying special attention to condition, medication regimen, age, and other demographic factors. Information on adverse events and associated events or conditions under which adverse events occurred, frequency, number of encounters, as well as duration or intensity, may be sent to an adverse reaction database to be used for medical research in addition to being noted in the treatment record of a care recipient.

Machine learning models may enable healthcare professionals and care providers to become aware of adverse events that present themselves, as well as provide recommendations for alternate treatment options. For instance, a machine learning model may suggest alternative medications based on adverse events experienced by a care recipient, based on which factors lead to bad outcomes for a similar care recipient as opposed to good outcomes for another care recipient, or recommend a change in the frequency or dosage of the medication being administered, e.g., instead of taking two pills together, take them with a time difference of 30 minutes.

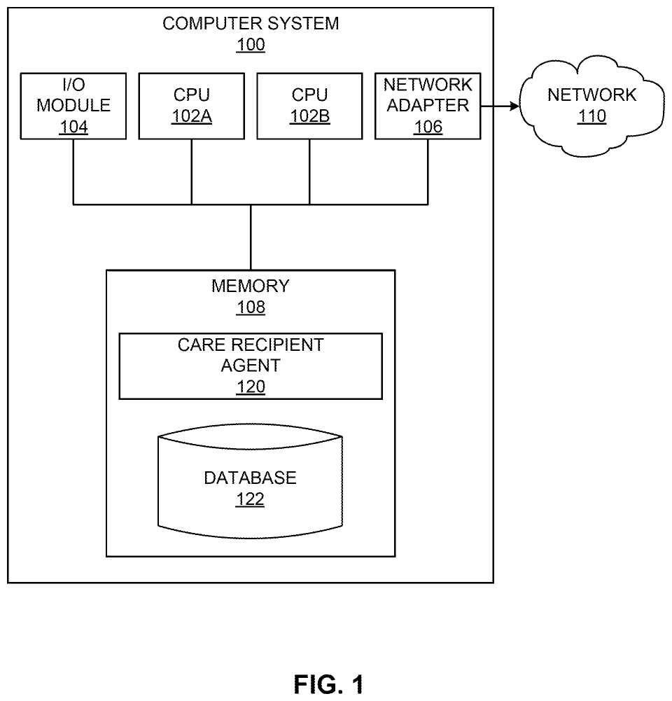

Referring now to FIG. 1, a block diagram is depicted illustrating the components of a computer system 100 that may be used to implement an artificial intelligence (AI) assistant agent for managing treatment in accordance with an embodiment. It should be appreciated that FIG. 1 provides only an illustration of one implementation and does not imply any limitations with regard to the environments in which different embodiments may be implemented. Many modifications to the depicted environments may be made based on design and implementation requirements.

The computer system 100 may be representative of any electronic device capable of executing machine-readable program instructions. The computer system 100 may be representative of a smartphone, a computer system, PDA, or other electronic devices. Examples of computing systems, environments, and/or configurations that may represented by the computer system 100 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

Computer system 100 may include one or more processors (CPUs) 102A-B, input/output circuitry 104, network adapter 106 and memory 108. CPUs 102A-B execute program instructions in order to carry out the functions of the present communications systems and methods. FIG. 1 illustrates an embodiment in which computer system 100 is implemented as a single multi-processor computer system, in which multiple CPUs 102A-B share system resources, such as memory 108, input/output circuitry 104, and network adapter 106. However, the present communications systems and methods also include embodiments in which computer system 100 is implemented as a plurality of networked computer systems, which may be single-processor computer systems, multi-processor computer systems, or a mix thereof. Input/output circuitry 104 provides the capability to input data to, or output data from, computer system 100. Network adapter 106 interfaces computer system 100 with a network 110, which may be any public or proprietary LAN or WAN, including, but not limited to the Internet.

Memory 108 stores program instructions that are executed by, and data that are used and processed by, CPU 102A-B to perform the functions of computer system 100. Memory 108 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an Integrated Drive Electronics (IDE) interface, or a variation or enhancement thereof, such as enhanced IDE (EIDE) or Ultra-Direct Memory Access (UDMA), or a Small Computer System Interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a Fibre Channel-Arbitrated Loop (FC-AL) interface.

The contents of memory 108 may vary depending upon the function that computer system 100 is programmed to perform. In the example shown in FIG. 1, example memory contents are shown representing routines and data for embodiments of the processes described herein. However, it may be recognized that these routines, along with the memory contents related to those routines, may not be included on one system or device, but rather may be distributed among a plurality of systems or devices, based on well-known engineering considerations. The present communications systems and methods may include any and all such arrangements.

Included within memory 108 may be the care recipient agent 120 which may run the routines that are described in the embodiments below. In order to store the treatment record for a care recipient along with other information that may be relevant to determination of the severity level of adverse events detected in biometric data, the care recipient agent 120 may access a database 122 that may store any information as described below. Also, as the care recipient agent 120 interacts with new captured biometric data, it may update the database 122 to include the information that it determines is relevant. The database 122 may be in any form that holds necessary information about the ongoing treatment of the care recipient.

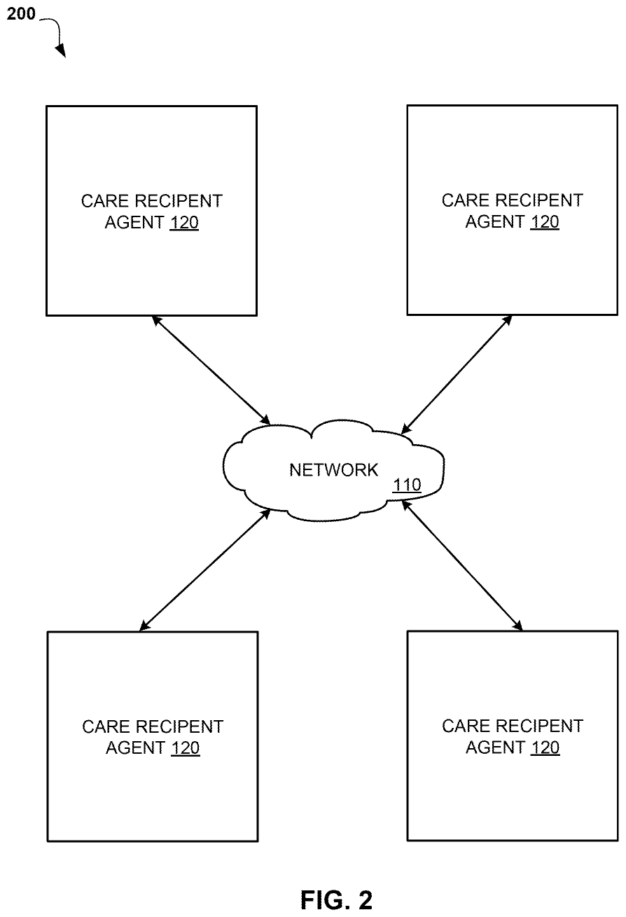

Referring to FIG. 2, a block diagram of a networked computing environment that may be used to form a group of care recipient agents 120 for the purpose of managing treatment and providing care to care recipients is depicted, according to at least one embodiment. The networked computer environment 200 may include a care recipient agent 120 associated with a local care recipient and at least one additional care recipient agent 120. The care recipient agents 120 may form an ad-hoc network with the local care recipient agent 120 and may be interconnected via a communication network 110. One of ordinary skill in the art may appreciate that the networked computer environment 200 may include a plurality of additional care recipient agents 120 but only three are shown in the configuration of FIG. 2 for illustrative brevity.

The communication network 110 may include various types of communication networks, such as a wide area network (WAN), local area network (LAN), a telecommunication network, a wireless network, a public switched network and/or a satellite network. The communication network 110 may include connections, such as wire, wireless communication links, or fiber optic cables. The network 110 may also include additional hardware not shown such as routers, firewalls, switches, gateway computers and/or edge servers. It may be appreciated that FIG. 2 provides only an illustration of one implementation and does not imply any limitations with regard to the environments in which different embodiments may be implemented. Many modifications to the depicted environments may be made based on design and implementation requirements. Accordingly, the communication network 110 may represent any communication pathway between the various components of the networked computer environment 200.

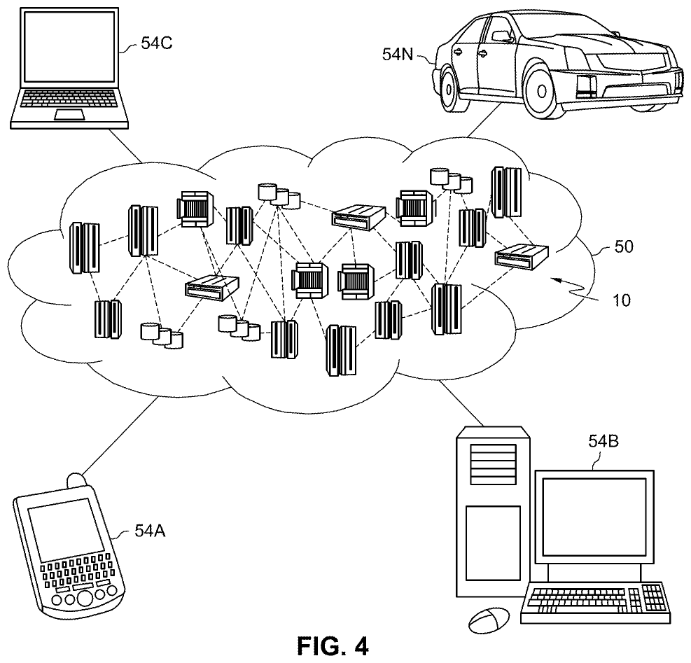

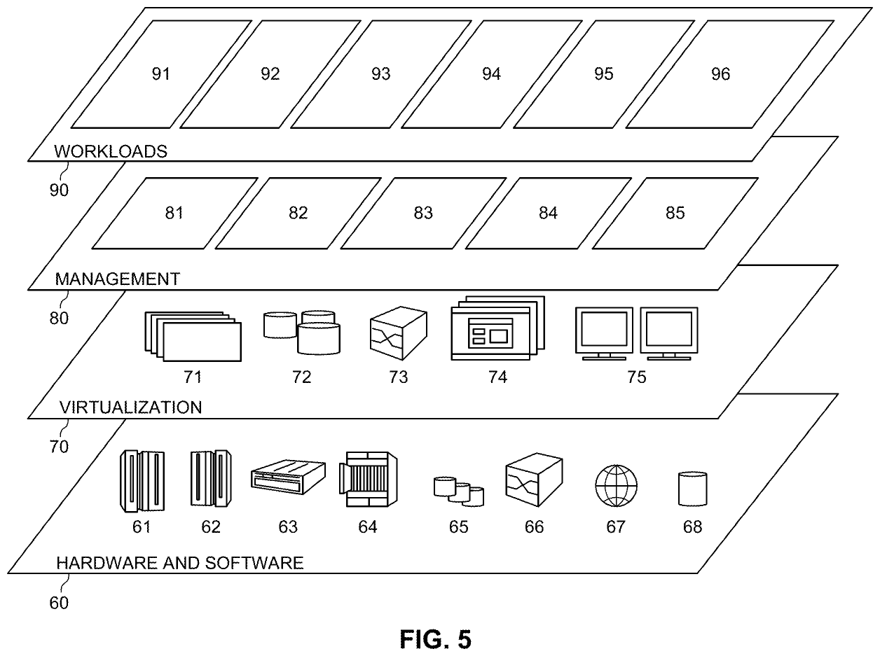

Care recipient agent 120 may be embedded in, for example, a mobile device, a telephone, a personal digital assistant, a netbook, a laptop computer, a tablet computer, a desktop computer, or any type of computing device capable of running a program and accessing a network. As will be discussed with reference to FIGS. 4 and 5, the care recipient agent 120 may also operate in a cloud computing service model, such as Software as a Service (SaaS), Platform as a Service (PaaS), or Infrastructure as a Service (IaaS). The servers may also be located in a cloud computing deployment model, such as a private cloud, community cloud, public cloud, or hybrid cloud.

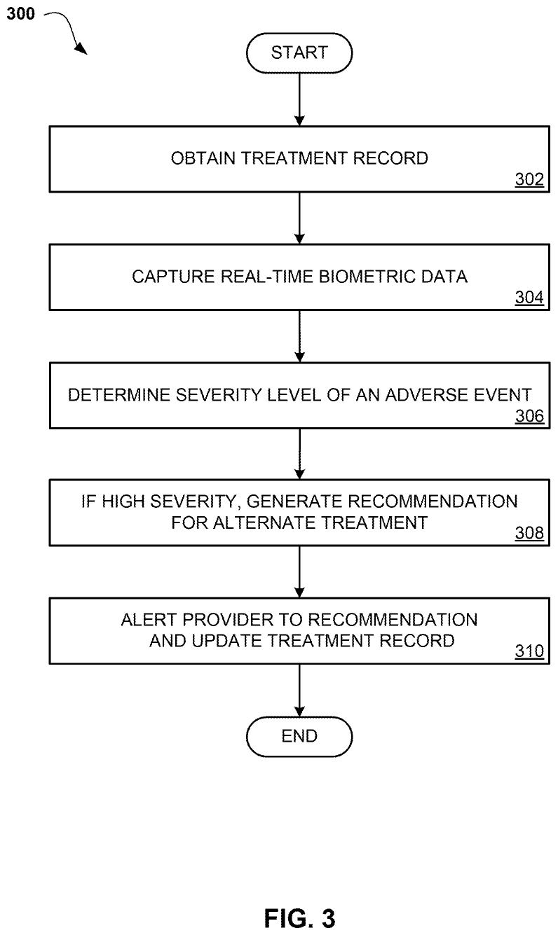

In the example shown in FIG. 2, a care recipient may visit a medical care provider, such as the office of a primary care physician or medical specialist, or the care recipient may be admitted to a hospital or urgent care center if an emergency is present. At the point of care, information may be collected from the care recipient that is added to a medical profile. For instance, a care recipient may be feeling shortness of breath and decide to visit an emergency room. The care provider may determine the identity of the patient and their vital signs as well as a medical history of all treatments currently being administered to the care recipient. While humans may interface with the care recipient directly, the data may also be entered into an automated care recipient agent 120 that may use artificial intelligence (AI) to manage the present and future care of the care recipient, as described further with respect to FIG. 3.

The care recipient agent may then communicate over the network 110 to locate other care recipient agents 120 that may be managing care for other care recipients and form a group of care recipient agents 120 with those care recipient agents 120 that have a similar profile. Examples of similarities may include demographic matches such as age or gender but may also include similar medications taken or treatment, as well as the same or similar medical conditions or underlying symptoms. Once the care recipient agent 120 discovers a set of care recipient agents 120 with the appropriate similarities, the care recipient agent 120 may form a group of care recipient agents that may be used to compare current adverse events that may be discovered in the local care recipient to past adverse events of care recipients in the group or to any biometric data that may be present in the group. The goal of forming the group is to discover adverse events and alternative treatments that may have been successful in treating care recipients that are similar to the local care recipient but were not easily discoverable by the local care recipient agent 120 or their care provider. One of ordinary skill in the art may recognize that the value of a group of care recipient agents 120 may extend beyond instant care decisions or adjustments but may also be used in medical research to document medical records of care recipients in diverse demographic groups and learn about the effects of various treatment options on those care recipients. Such research may be used to refine the machine learning models in use and provide feedback and further information to care providers. However, it should be noted that any information that may be used for general research must be anonymous and not identify the specific care recipient. In addition, any medical information of a specific care recipient, whether or not it is anonymous, requires the consent of the care recipient.

Referring to FIG. 3, an operational flowchart illustrating a process 300 for managing care recipient treatment options, including medication regimens, is depicted according to at least one embodiment. At 302, records of a care recipient treatment may be obtained from a server, including details about the care recipient such as demographics, as well as medical data associated with the care recipient. The medical data associated with the care recipient may include prior and/or ongoing conditions that may or may not relate to the current treatment and may serve as baseline data for the care recipient that may be used when analyzing events that occur during the current treatment. For instance, if a care recipient is known to have arthritis in certain joints, then pain detected in the same joints may be considered less severe than pain detected in other areas or than pain detected in other care recipients. The treatment record may also include details of a current treatment such as known side effects of a medication or other common events in care recipients that may be reported through clinical trials or other medical studies.

At 304, real-time biometric data may be collected from the care recipient using a variety of sensors. For example, a wearable mobile device such as a smartwatch or smartphone may be equipped to detect heart rate, blood pressure and/or patterns of breathing, as well as muscle movements throughout the body. If the care recipient is in a medical care facility, whether admitted to a hospital or visiting a physician or other care provider, there may be dedicated devices physically attached to the care recipient for the purpose of capturing the real-time biometric data. One of ordinary skill in the art may recognize that there are several ways to capture biometric data depending on the location of the care recipient. This biometric data may be used to determine a physiological state of the care recipient, taking care to monitor for unexpected or adverse events.

It is important to note that any real-time monitoring of a care recipient as mentioned herein requires the informed consent of all those people whose biometric data is captured for analysis. Consent may be obtained in real time or through a prior waiver or other process that informs a subject that their biometric data may be captured by sensors or other sensitive personal data may be gathered through any means and that this data may be analyzed by any of the many algorithms that may be implemented herein. A care recipient may opt out of any portion of the real-time monitoring at any time.

In addition to determining a physiological state, biometric sensor data that may be captured may be used to determine an emotional state of a care recipient (e.g., by using the biometric sensor data to predict an emotion of the care recipient). This emotional state may be equally as valuable as the physiological state, i.e., an objective medical state that is determined from biometric data, in determining the proper course of medical treatment. Examples of an emotional state may include a state of mind or any other information about the state of a care recipient that may not be determined with biometric data such as a care recipient wincing in pain or crying out in anger or pain or displaying an emotion that may indicate the care recipient's current medical condition or physiological state. To determine the emotional state of a care recipient, any of several techniques may be used to analyze the biometric data that has been captured. For instance, image recognition algorithms may be used with extracted images from captured video of the care recipient to determine specific body language or other known visual cues such as facial expressions or eye movements to determine a participant's current state of mind.

Automatic speech recognition (ASR) techniques in conjunction with speech-to-text (STT) and natural language processing (NLP) algorithms may also be used to analyze possible captured audio from the area near the care recipient, including possible conversations with a care provider, to determine the emotional state of the care recipient. For instance, certain spoken keywords or phrases, such as “I feel pain in my stomach” or “I have been feeling light-headed since I started this medication,” may indicate the state of the care recipient in a way that may not be detected by the biometric sensors.

In addition, any captured audio may be used to determine a tone of voice or inflection in use by the care recipient, which may be useful in determining the emotion of the care recipient, since a raised inflection or a halted pattern in the care recipient's speech may indicate severe pain being suffered by the care recipient. In an embodiment, a supervised machine learning classification model may be trained to predict the tone or inflection of the voice in spoken audio. One or more of the following machine learning algorithms may be used: logistic regression, naive Bayes, support vector machines, deep neural networks, random forest, decision tree, gradient-boosted tree, multilayer perceptron, and one-vs-rest. In an embodiment, an ensemble machine learning technique may be employed that uses multiple machine learning algorithms together to assure better prediction when compared with the prediction of a single machine learning algorithm. In this embodiment, training data for the model may include several audio samples from a variety of users expressing various inflections and tones of voice. The training data may be captured from a single user or a group of users, with user consent required prior to capture. The classification results may be stored in a database so that the data is most current, and the output would always be up to date.

One of ordinary skill in the art will recognize that a supervised machine learning classification model such as described above may also be used to predict the emotional state of the care recipient. In fact, there may be multiple machine learning models used in the management of the care recipient's treatment and the training data for these models may be shared between the models or not. In addition, the same model may be used to generate multiple predictions. As an example, a prediction of tone of voice or inflection may use the same type of model as a prediction of a care recipient's emotional state. It is not required that every machine learning model is separate or combined, other than incompatible model types, for instance a classification model would not be used for a result that is not a classification.

At 306, an adverse event may be detected in the biometric data and a severity level of the adverse event may be determined using a machine learning model. An adverse event may be defined as any event that may be detrimental to the care recipient, e.g., blood pressure or heart rate readings that are too high or too low or any other uncommon physiological signs such as an abnormal heartbeat. To determine the severity of an adverse event, the detected adverse event may be compared to the baseline data of the care recipient obtained in 302. If there are specific reasons why a care recipient may experience certain symptoms, e.g., the care recipient may be taking medication not related to the treatment that presents certain symptoms or the care recipient may have an abnormal heartbeat at rest, then the severity level of the adverse event may be low or the adverse event may even be ignored.

Adverse events that may be detected may also be compared to details about the medical treatment administered to the care recipient to determine if the adverse event may have been recorded in the past as a known side effect of that treatment. For instance, some medications may have a common side effect such as lowering blood pressure. If the biometric data of the care recipient includes low blood pressure measurements, while this may be generally concerning, low blood pressure may already be expected and may lower the severity level of the adverse event. At the same time, if a care recipient experiences an adverse event that may not be known in the treatment record or may be of greater intensity than seen in the treatment record, then the severity level may be high.

The severity level may be expressed as a range of values, such that a portion of the range of values may be considered low and not requiring any further action but should the severity level rise or be above a certain threshold, then further analysis may be required, and action may need to be taken. A threshold (e.g., a range of values which may require further action) may be defined and/or updated (e.g., maintained) by a human care provider. One of ordinary skill in the art may also recognize that there are multiple ways of expressing severity as an objective value and it is not required that the severity level be expressed as a number. It is only required that adverse events that may be explained or considered slight be filtered out and that analysis and action may be taken on adverse events with a minimum severity level.

In an embodiment, the assignment of the severity level may be a collaboration between the care recipient and a human care provider and feedback may be applied to adjust the severity level as further information is determined. For instance, in the example above where lowered blood pressure may be an indicated side effect of a medication, the known information may be used as training data for a supervised machine learning model, such as described above, that may adjust the severity level based on the known information. However, any possible adjustment may be subject to approval of a human care provider such that the adjustment may be overridden on a case by case basis.

At 308, once an adverse event that may be experienced by a care recipient has a severity level assigned that is above the threshold described above, a recommendation for alternative treatment may be generated. To accomplish this task, a group of care recipient agents 120 such as the group that may be formed in FIG. 2 above may be queried to locate care recipients to which the local care recipient's biometric data and adverse event may be compared. For instance, if the care recipient has reported shortness of breath or blood pressure readings from the biometric data indicate numbers that are high, both of which may indicate a high severity level, then there may be other care recipients who have similar medical conditions or are taking the same medication who also have these symptoms. As described in FIG. 2, the care recipient agent 120 may already have formed a group of care recipient agents that may include this data. The care recipient agent 120 may discover this data and also any possible adjustments to treatment of the other care recipients that may have been made to correct for the same or similar adverse event. From this process, any information that is discovered about the care recipient that may be used to assist other care recipients may be used as training data for yet another supervised machine learning model that may recommend an alternative treatment for the care recipient.

At 310, a notification comprising the adverse event that may be detected and determined to have a high severity level and a possible recommendation for an alternative medication or alternative regimen for administering the medical treatment may be used to alert a medical professional, who may determine if the recommendation is appropriate and take action as necessary. One of ordinary skill in the art may recognize that any action that may be taken by a care recipient agent 120 is subject to the consent of the care recipient and the approval of a duly licensed medical care professional. The notification that is generated in 310 may take the form of a message to the care provider or care recipient but may also be in the form of a report to the care provider that includes the details of the agent's analysis and also the information that may have been received from the group of care recipient agents 120 such that the care provider may inspect all the information that the care recipient agent possesses and make an independent determination of the proper treatment course to follow, e.g., a change to the dosage or frequency of the medication, a different medication or a different treatment altogether.

In addition to notifying the care provider, the treatment record of the care recipient may be updated to include a recommendation of the care recipient agent 120, along with a clear indication (e.g., disclaimer) that the recommendation is that of the care recipient agent 120 and not that of any human care provider, as well as the eventual course of treatment that is followed by the care provider, for use by the other care recipient agents 120 that are currently present in the group of agents or those agents 120 that form new groups with the care recipient agents 120.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 4 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66, such as a load balancer. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include mapping and navigation 91; software development and lifecycle management 92; virtual classroom education delivery 93; data analytics processing 94; transaction processing 95; and care recipient agent applications 96. Care recipient agent applications may refer to forming a group of care recipient agents to share information and manage treatment of care recipients.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for managing a medical treatment of a care recipient, the method comprising:
obtaining a treatment record for the care recipient from a server;
capturing real-time biometric data of the care recipient using a plurality of sensors;
in response to detecting an adverse event in the real-time biometric data, determining a severity level of the adverse event by comparing the real-time biometric data with the treatment record;
in response to the severity level being above a threshold, generating a recommendation for an alternative treatment; and
transmitting a notification comprising the recommendation to a treatment provider.

2. The computer-implemented method of claim 1, further comprising:
receiving a plurality of care recipient agents, wherein each care recipient agent includes a medical record of a respective care recipient;
selecting a care recipient agent from the plurality of care recipient agents by comparing the real-time biometric data and the treatment record of the care recipient to the medical record of each care recipient agent; and
placing the selected care recipient agent in a group of care recipient agents.

3. The computer-implemented method of claim 1, wherein capturing the real-time biometric data includes using a machine learning model that predicts a user's emotions from the captured biometric data to determine an emotional state of the care recipient.

4. The computer-implemented method of claim 2, wherein determining the severity level of the adverse event further comprises comparing the captured real-time biometric data and the treatment record of the care recipient to the medical record of other care recipient agents within the group of care recipient agents.

5. The computer-implemented method of claim 1, wherein transmitting the notification further comprises updating the treatment record of the care recipient with the alternative treatment.

6. The computer-implemented method of claim 1, wherein the alternative treatment comprises a change to a medication currently used in the medical treatment.

7. The computer-implemented method of claim 1, wherein the alternative treatment comprises a change to a current administration of the medical treatment.

8. A computer system for managing a medical treatment of a care recipient comprising:
one or more processors, one or more computer-readable memories, one or more computer-readable tangible storage media, and program instructions stored on at least one of the one or more tangible storage media for execution by at least one of the one or more processors via at least one of the one or more memories, wherein the computer system is capable of performing a method comprising:
obtaining a treatment record for the care recipient from a server;
capturing real-time biometric data of the care recipient using a plurality of sensors;
in response to detecting an adverse event in the real-time biometric data, determining a severity level of the adverse event by comparing the real-time biometric data with the treatment record;
in response to the severity level being above a threshold, generating a recommendation for an alternative treatment; and
transmitting a notification comprising the recommendation to a treatment provider.

9. The computer system of claim 8, further comprising:
receiving a plurality of care recipient agents, wherein each care recipient agent includes a medical record of a respective care recipient;
selecting a care recipient agent from the plurality of care recipient agents by comparing the real-time biometric data and the treatment record of the care recipient to the medical record of each care recipient agent; and
placing the selected care recipient agent in a group of care recipient agents.

10. The computer system of claim 8, wherein capturing the real-time biometric data includes using a machine learning model that predicts a user's emotions from the captured biometric data to determine an emotional state of the care recipient.

11. The computer system of claim 9, wherein determining the severity level of the adverse event further comprises comparing the captured real-time biometric data and the treatment record of the care recipient to the medical record of other care recipient agents within the group of care recipient agents.

12. The computer system of claim 8, wherein transmitting the notification further comprises updating the treatment record of the care recipient with the alternative treatment.

13. The computer system of claim 8, wherein the alternative treatment comprises a change to a medication currently used in the medical treatment.

14. The computer system of claim 8, wherein the alternative treatment comprises a change to a current administration of the medical treatment.

15. A computer program product for managing a medical treatment of a care recipient comprising:
a computer readable storage device having program instructions embodied therewith, the program instructions executable by a processor to cause the processor to perform a method comprising:
obtaining a treatment record for the care recipient from a server;
capturing real-time biometric data of the care recipient using a plurality of sensors;
in response to detecting an adverse event in the real-time biometric data, determining a severity level of the adverse event by comparing the real-time biometric data with the treatment record;
in response to the severity level being above a threshold, generating a recommendation for an alternative treatment; and
transmitting a notification comprising the recommendation to a treatment provider.

16. The computer program product of claim 15, further comprising:
receiving a plurality of care recipient agents, wherein each care recipient agent includes a medical record of a respective care recipient;
selecting a care recipient agent from the plurality of care recipient agents by comparing the real-time biometric data and the treatment record of the care recipient to the medical record of each care recipient agent; and
placing the selected care recipient agent in a group of care recipient agents.

17. The computer program product of claim 15, wherein capturing the real-time biometric data includes using a machine learning model that predicts a user's emotions from the captured biometric data to determine an emotional state of the care recipient.

18. The computer program product of claim 16, wherein determining the severity level of the adverse event further comprises comparing the captured real-time biometric data and the treatment record of the care recipient to the medical record of other care recipient agents within the group of care recipient agents.

19. The computer program product of claim 15, wherein transmitting the notification further comprises updating the treatment record of the care recipient with the alternative treatment.

20. The computer program product of claim 15, wherein the alternative treatment comprises a change to a medication currently used in the medical treatment.
